(19) United States
(12) Patent Application Publication
(10) Pub. No.: US 2015/0164704 A1
(43) Pub. Date: Jun. 18, 2015

(54) SANITARY NAPKIN

(71) Applicant: Ju Seok CHAE, Seongnam-si (KR)

(72) Inventor: Ju Seok CHAE, Seongnam-si (KR)

(21) Appl. No.: 14/541,381

(22) Filed: Nov. 14, 2014

(30) Foreign Application Priority Data
Dec. 16, 2013 (KR) 10-2013-0156068

(51) Int. Cl. A61F 13/47; A61F 13/472

(52) U.S. Cl. CPC A61F 13/4704; A61F 13/47236; A61F 2013/4512

(57) ABSTRACT

Provided is a sanitary napkin including a lift-up pad detached along a perforated line formed in a main pad and lifted up. The sanitary pad may drastically reduce leakage of menstrual blood to buttocks, and therefore greatly improve comfort of a woman's life, such as by providing a restful sleep without anxiety of the leakage of the menstrual blood.

### CROSS-REFERENCE TO RELATED APPLICATION

This application claims priority to and the benefit of Korean Patent Application No. 10-2013-0156068, filed on Dec. 16, 2013, the disclosure of which is incorporated herein by reference in its entirety.

### BACKGROUND

1. Field of the Invention

The present invention relates to a sanitary napkin, and more particularly, to a sanitary napkin having a reinforced leakage preventing function.

2. Discussion of Related Art

Generally, a sanitary napkin is a product for absorbing and containing female secretions such as menstrual blood, and recently, pad-type sanitary napkins attached to an inside of an undergarment have been most widely used.

However, when such a pad-type sanitary napkin is used, particularly during sleep, some of the menstrual blood frequently leaks toward the buttocks. That is, when a women is lying down as when sleeping, gravity is applied toward the buttocks, and thus the menstrual blood leaks easily, and if the woman turns while sleeping, a gap between the pad and the buttocks becomes larger, and thus a possibility of the menstrual blood leaking to the gap between the pad and the buttocks increases. Therefore, clothes or bedding can be soiled by the leaked menstrual blood, thereby causing inconvenience in daily life, and therefore measures for this are required.

### SUMMARY OF THE INVENTION

The present invention is directed to providing a sanitary napkin that is improved to effectively reduce a probability of leakage of menstrual blood.

In one aspect, the present invention provides a sanitary napkin, which includes a main pad, and a lift-up pad prepared to be lifted up along a perforated line formed in the main pad.

The lift-up pad is formed to be placed at a gluteal cleft.

An auxiliary pad can be attached on an opposite side of the main pad on which the lift-up pad is formed.

The lift-up pad has a tab capable of being used to detach the lift-up pad along the perforated line.

The main pad includes an absorbing body for absorbing menstrual blood, a soft cover covering a top body-contacting surface of the absorbing body, and a waterproof film covering an opposite side of the soft cover.

The opposite side of the body-contacting surface of the lift-up pad is covered with a waterproof film.

A perforation controlling part in which a perforating interval is relatively long is designed in the middle of the perforated line.

The lift-up pad is formed in any one of a straight line, a straight line with wings, and a dovetail shape.

Use without detachment of the lift-up pad along the perforated line is possible.

### BRIEF DESCRIPTION OF THE DRAWINGS

The above and other objects, features, and advantages of the present invention will become more apparent to those of ordinary skill in the art by describing in detail exemplary embodiments thereof with reference to the adhered drawings, in which:

FIG. 1 is an isolated perspective view of a structure of a sanitary napkin according to an exemplary embodiment of the present invention;

FIG. 2 is a perspective view of a state of the sanitary napkin shown in FIG. 1 before use;

FIG. 3 is a perspective view of a state of the sanitary napkin of FIG. 1 in use;

FIG. 4 is a perspective view of an opposite side of a lift-up pad of the sanitary napkin of FIG. 1; and

FIGS. 5A to 5C are diagrams of modified examples of the sanitary napkin of FIG. 1.

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the invention

Hereinafter, exemplary embodiments of the present invention will be described in more detail with reference to the appended drawings.

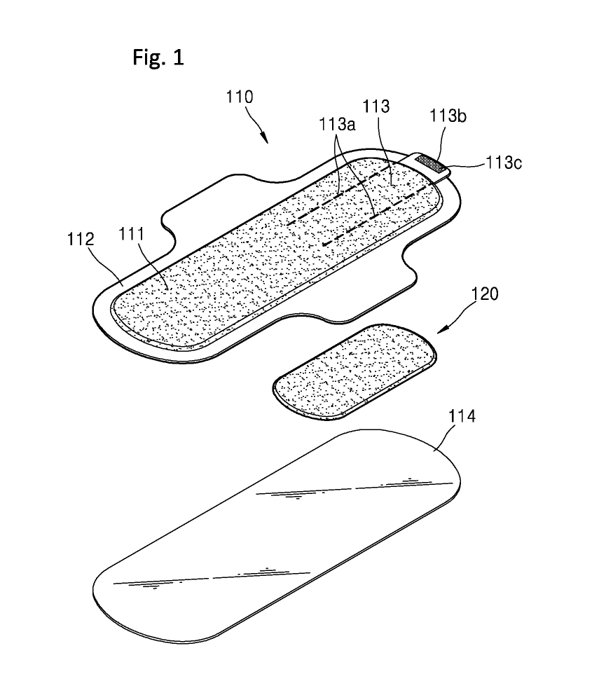

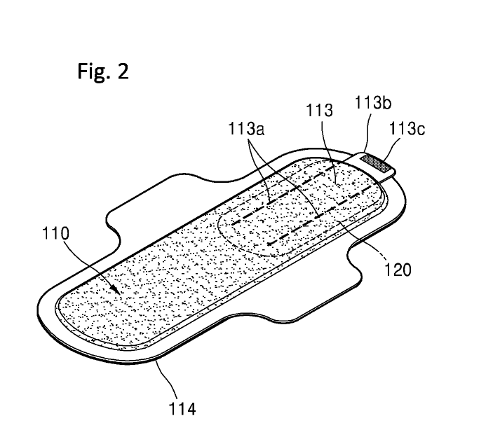

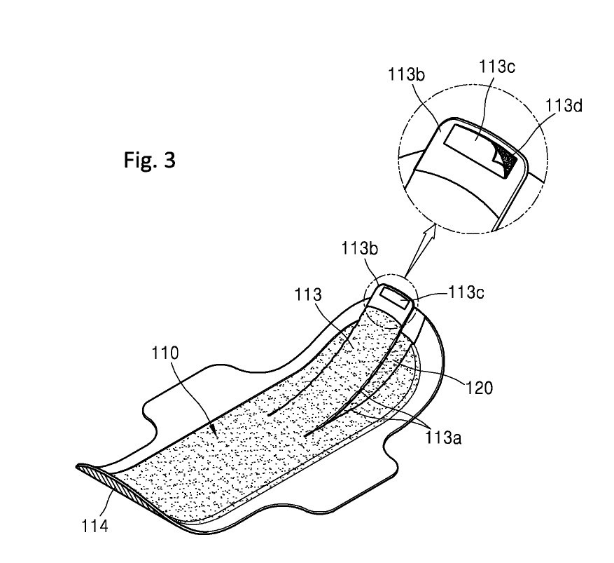

FIG. 1 is an isolated perspective view of a sanitary napkin according to an exemplary embodiment of the present invention, and FIGS. 2 and 3 are views of states before and during use.

Referring to FIGS. 1 to 3, a sanitary napkin of the exemplary embodiment includes a main pad 110, a lift-up pad 113 and an auxiliary pad 120.

First, the main pad 110 includes an absorbing body 111 for absorbing menstrual blood, a soft cover 112 covering a top body-contacting surface of the absorbing body 111, and a waterproof film 114 covering an opposite side of the soft cover 112. Accordingly, when the sanitary napkin is worn, the menstrual blood is absorbed by the absorbing body 111 through the soft cover 112, and the waterproof film 114 serves to prevent the absorbed menstrual blood from staining an undergarment.

In addition, the lift-up pad 113 is formed along a perforated line 113a on a back side of the main pad 110. That is, the lift-up pad 113 is a part that is detached from the main pad 110 along the perforated line 113a and lifted up, and when detached and lifted up as shown in FIG. 3, the pad is placed at a gluteal cleft of a wearer.

Reference numeral 113b refers to a tab, which is designed to be grasped when the user detaches the lift-up pad 113 along the perforated line 113a, and has an attached surface 113d that can be attached to a body after a releasing tape 113c of the tab is removed. Accordingly, the lift-up pad 113 is detached along the perforated line 113a, lifted up, and placed at the gluteal cleft, and the attached surface 113d can be stably worn by being attached to a coccygeal region.

The auxiliary pad 120 serves to auxiliarily protect an opposite side of the lift-up pad 113 after the lift-up pad 113 is detached and lifted up. That is, when there is no auxiliary pad 120, a back side becomes empty after the lift-up pad 113 is detached and lifted up. As the auxiliary pad 120 is equipped, the danger of leakage is doubly prevented.

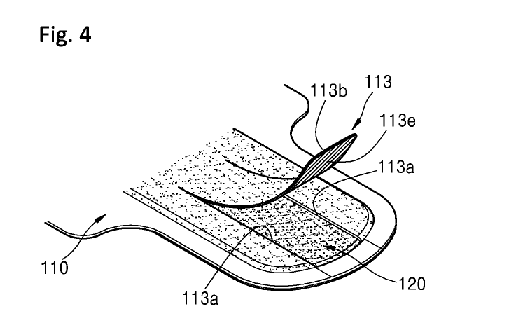

In addition, as shown in FIG. 4, a waterproof film 113e such as the waterproof film 114 of the entire main pad 110 may be attached to the opposite side of the lift-up pad 113.

The sanitary napkin having such a structure may be used as follows. First, before use, the main pad 110 and the lift-up pad 113 of the sanitary napkin are integrated as shown in FIG. 2. When it is considered unnecessary to detach the lift-up pad 113 before use, the sanitary napkin may be used without detaching the lift-up pad 113.

However, when more secure prevention of leakage of the menstrual blood to the buttocks occurring in bed is desired, the lift-up pad 113 is detached along the perforated line 113a by grasping the tab 113b and lifting it up before wearing, as shown in FIG. 3. After the lift-up pad 113 is placed at the gluteal cleft, hardly any menstrual blood leaks toward the buttocks. Here, when the releasing tape 113c is detached, and the attached surface 113d is attached to the coccygeal region, a more stable wearing state may be maintained.

Accordingly, when the lift-up pad 113 is detached in use as needed, the probability of the menstrual blood leaking toward the buttocks while sleeping may be sufficiently decreased.

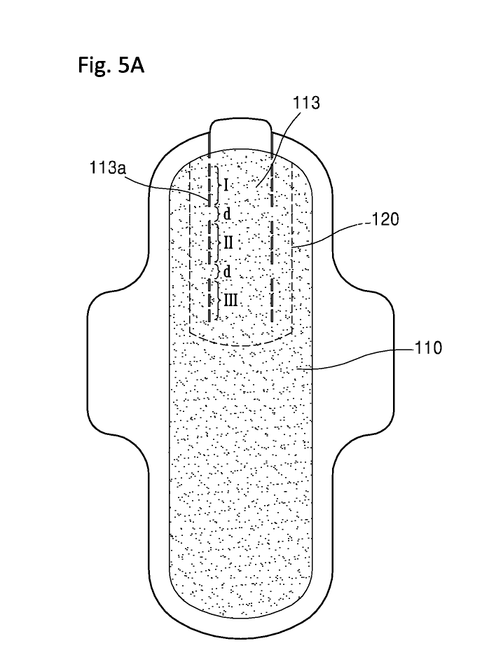

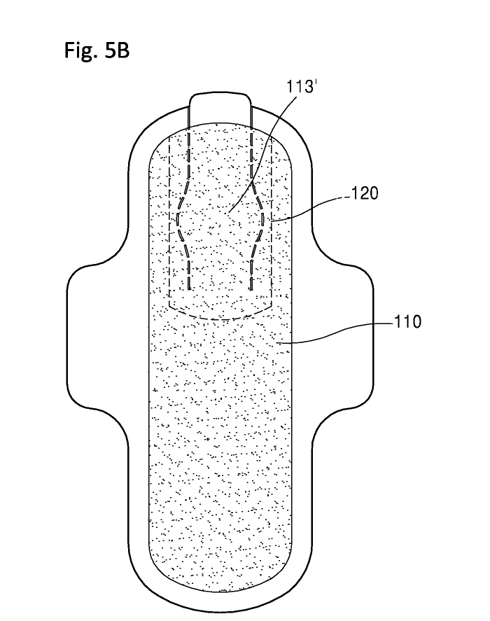

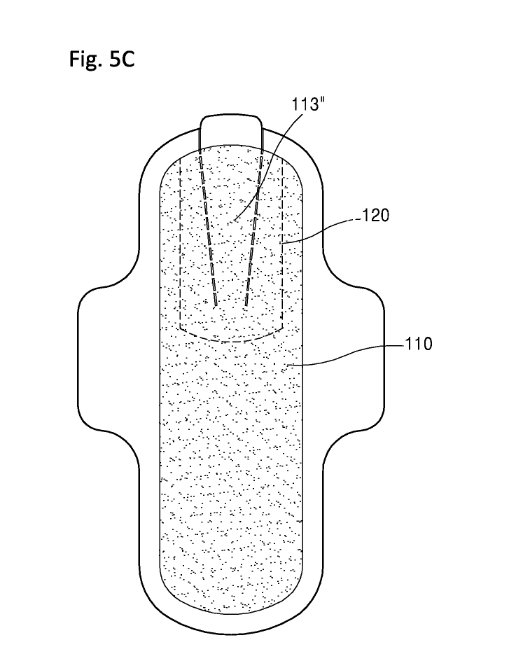

Meanwhile, the sanitary napkin of the above embodiment may be modified to various structures as shown in FIGS. 5A to 5C.

First, in the above-described embodiment, the case in which the perforated line 113a is formed at a regular interval on the main pad 110 is exemplified, but it may be modified into a structure in which a perforation controlling part d having a relatively long perforated interval in the middle as shown in FIG. 5A. Therefore, when the lift-up pad 113 is detached, the perforation stops after being detached to a first perforated line I, and stops again after being detached to a second perforated line II. Since a length of lifting up the lift-up pad 113 may be changed according to a body size of the user, the perforation controlling part d is made for the user to easily control the perforation midway. Accordingly, for a user who should detach a long portion of the lift-up pad 113, the lift-up pad 113 is detached and lifted up to a third perforated line III, and for a user who should detach a short portion of the lift-up pad 113, the lift-up pad 113 is detached and lifted up to the first perforated line I.

That is, the sanitary napkin of the present invention includes the perforation controlling part d, and thus can be easily used by a wide range of users regardless of their age or body size.

In addition, while a shape of the perforated line 113a, that is, a shape of the lift-up pad 113, is a straight line in the above-described embodiment, to the shape may be a straight line with wings 113′ as shown in FIG. 5B or the shape of a dovetail 113″. That is, the shape of the lift-up pad 113 may be modified in various shapes without limitation as long as the lift-up pad 113 is placed at the gluteal cleft.

Therefore, the sanitary napkin as described above drastically reduces the leakage of the menstrual blood to the lift-up pad 113, and can greatly improve comfort of a woman's life, such as by providing restful sleep without anxiety of the leakage of the menstrual blood.

Moreover, the sanitary napkin can absorb a great amount of the menstrual blood using the lift-up pad 113. Therefore, even when a relatively thin sanitary napkin as described herein is worn, the same effect as wearing a conventional thick sanitary napkin may be obtained.

Accordingly, when the sanitary napkin of the present invention is worn, even when wearing tight clothes such as leggings, the shape of the sanitary napkin is hardly visible from behind a wearer.

A sanitary napkin having a configuration as described herein can drastically reduce leakage of menstrual blood to the buttocks, and can greatly improve comfort of a woman's life, such as by providing restful sleep without anxiety of the leakage of the menstrual blood.

The invention has been described with reference to an exemplary embodiment illustrated in the drawings, but has been merely exemplified. It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. A sanitary napkin, comprising:
a main pad; and
a lift-up pad designed to be detached along a perforated line formed in the main pad and lifted up.

2. The sanitary napkin according to claim 1, wherein the lift-up pad is formed to be placed at a gluteal cleft.

3. The sanitary napkin according to claim 1, further comprising:
an auxiliary pad attached on an opposite side of the main pad on which the lift-up pad is formed.

4. The sanitary napkin according to claim 1, wherein the lift-up pad has a tab capable of being used to detach the lift-up pad along the perforated line.

5. The sanitary napkin according to claim 4, wherein the tab has an adhesive surface capable of being attached to a body.

6. The sanitary napkin according to claim 1, wherein the main pad includes an absorbing body for absorbing menstrual blood, a soft cover covering a top body-contacting surface of the absorbing body, and a waterproof film covering an opposite side of the soft cover.

7. The sanitary napkin according to claim 6, wherein the opposite side of the body-contacting surface of the lift-up pad is covered with a waterproof film.

8. The sanitary napkin according to claim 1, wherein a perforation controlling part in which a perforating interval is relatively long is designed in the middle of the perforated line.

9. The sanitary napkin according to claim 1, wherein the lift-up pad is formed in any one of a straight line, a straight line with wings, and a dovetail shape.

10. The sanitary napkin according to claim 1, wherein use without detachment of the lift-up pad along the perforated line is possible.
